(19) United States
(12) Patent Application Publication
(10) Pub. No.: US 2011/0275707 A1
(43) Pub. Date: Nov. 10, 2011

(54) SUBSTITUTED AFPO (6-ARYL-4H-FURO[3,2-C]PYRAN-4-ONE) DERIVATIVES AS ANTI-CANCER AGENTS

(76) Inventors: Kuo-Hsiung Lee, Chapel Hill, NC (US); Yizhou Dong, Chapel Hill, NC (US); Qian Shi, Chapel Hill, NC (US); Kenneth F. Bastow, Chapel Hill, NC (US); Pei-Chi Wu, Durham, NC (US)

(21) Appl. No.: 13/140,330

(22) PCT Filed: Dec. 16, 2009
(86) PCT No.: PCT/US09/68165

§ 371 Date: Jul. 22, 2011

Related U.S. Application Data
(62) Division of application No. 61/139,241, filed on Dec. 19, 2008.

(51) Int. Cl. A61K 31/366; A61P 35/00; A61P 35/02; C07D 493/04

(52) U.S. Cl. 514/456; 549/283

(57) ABSTRACT

Compounds of Formulas I are described, along with methods of using such compounds for the treatment of cancer and pharmaceutical formulations thereof.

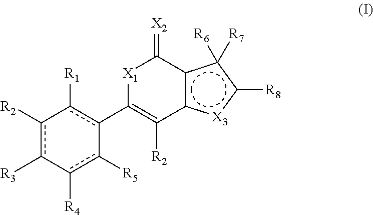

### STATEMENT OF GOVERNMENT SUPPORT

This invention was made with government support under NIH grant CA 17625. The Government has certain rights to this invention.

### FIELD OF THE INVENTION

The present invention concerns active compounds, formulations thereof, and methods of use thereof, particularly in methods of treating cancer.

### BACKGROUND OF THE INVENTION

Breast cancer is the most common cancer among women. (Albrand, G.; Terret, C. Drugs Aging 2008, 25, 35-45). According to the American Cancer Society, the disease accounts for more than one quarter of cancers diagnosed in US women. In 2007, it was estimated that 18,000 new cases of invasive breast cancer would be diagnosed in women, as well as an estimated 60,000 additional cases of in situ breast cancer. (Breast Cancer Facts & Figures, 2008).

Most clinically used anticancer drugs cause general toxicity to proliferating cells, which can severely limit their therapeutic value. (Chari, R. V. J. Acc. Chem. Res. 2008, 41, 98-107). Thus, much effort has been made to increase tissue, cell, and target selectivity for chemotherapy. (Pedram, B.; van Oeveren, A.; Mais, D. E.; Marschke, K. B.; Verbost, P. M.; Groen, M. B.; Zhi, L. J. Med. Chem. 2008, 51, 3696-3699). However, although new cytotoxic agents with unique mechanisms of action have been developed continuously, many of them have not been therapeutically useful due to low tumor selectivity. These facts prompted us to design and develop novel potent and selective anti-breast cancer agents.

Natural products have been the most important source of new medicinal leads. (Saklani, A.; Kutty, S. K. Drug Discov Today 2008, 13, 161-71). However, the structural complexity of natural products, such as intricate ring systems and numerous chiral centers, may lead to limited supplies and hamper mechanism of action studies and clinical development. (Magedov, I. V.; Manpadi, M.; Ogasawara, M. A.; Dhawan, A. S.; Rogelj, S.; Van Slambrouck, S.; Steelant, W. F. A.; Evdokimov, N. M.; Uglinskii, P. Y.; Elias, E. M.; Knee, E. J.; Tongwa, P.; Antipin, M. Y.; Kornienko, A. J. Med. Chem. 2008, 51, 2561-2570). Structural simplification of natural products is a powerful and highly productive tool for lead development and analog design. (Raghavan, B.; Balasubramanian, R.; Steele, J. C.; Sackett, D. L.; Fecik, R. A. J. Med. Chem. 2008, 51, 1530-1533). A well-known example is the simplification of morphine, which led to the clinically used medicines levophanol and meperidine. (Wolff, M. E. Burger's Medicinal Chemistry and Drug Discovery; fifth ed., 1994; Vol. 1). Neo-tanshinlactone (1) is a steroid-like tetracyclic natural product originally isolated from the traditional Chinese medicine Tanshen. Compound 1 and its first generation analogs (2) with various substitutions around the molecular scaffold have been totally synthesized and biologically studied. Compound 1 was reported as a highly selective inhibitor of the growth of breast cancer cell lines MCF-7 and SK-BR-3. (Wang, X.; Bastow, K. F.; Sun, C. M.; Lin, Y. L.; Yu, H. J.; Don, M. J.; Wu, T. S.; Nakamura, S.; Lee, K. H. J Med Chem 2004, 47, 5816-9; Wang, X.; Nakagawa-Goto, K.; Bastow, K. F.; Don, M. J.; Lin, Y. L.; Wu, T. S.; Lee, K. H. J Med Chem 2006, 49, 5631-4). It was speculated that the planarity of the molecular structure is important to the activity. However, how the skeletal planarity affects activity and selectivity, how the four individual rings contribute to activity, and how the activity and selectivity change if the tetracyclic molecule of 1 is simplified remained unanswered. In our recent study, we used chemical and biological strategies to investigate structurally simplified 1-analogs in order to answer these questions.

In our prior paper, we reported a study on how the individual A, C, and D rings influence in vitro anti-breast cancer activity. The results revealed a new class of potent and selective anti-breast cancer agents, 2-(furan-2-yl)-naphthalen-1-ol derivatives (3), which encouraged us to further simplify the scaffold of 1. Herein, we report a new chemical entity, substituted AFPO (6-aryl-4H-furo[3,2-c]pyran-4-one) derivatives (4), the synthesis of these 4-analogs, and their cytotoxic activity against several human tumor cell lines.

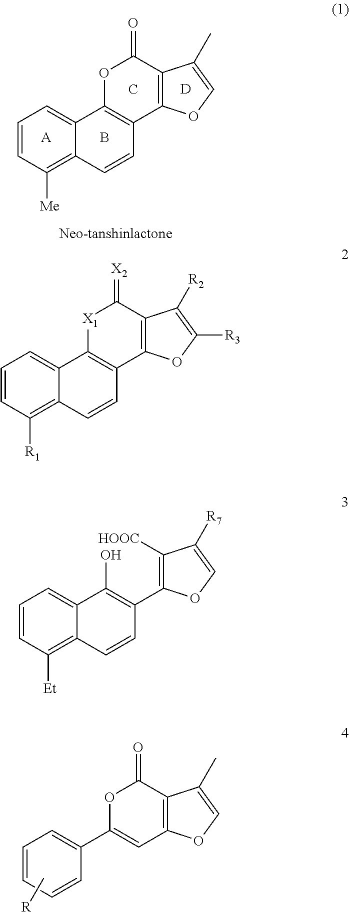

Scheme 1. Structures of neo-tanshinlactone (1), first generation neo-tanshinlactone analog 2, the second generation optimized analog 3, and a newly designed scaffold 4.

### SUMMARY OF THE INVENTION

A first aspect of the present invention is a compound of Formula I:

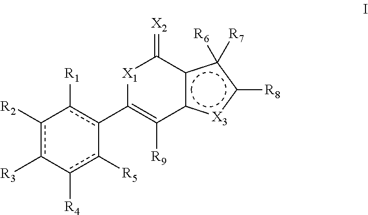

wherein:
R₁, R₂, R₃, R₄, R₅, R₆, R₇, R₈, and R₉ are each independently selected from the group consisting of H, lower alkyl, hydroxy, lower alkoxy, halo, amino, aminoalkyl, alkylamino, nitro, heteroaryl, aryl, OC(═O)R₁₄, OC(═O)OR₁₄, OC(═O)N(R₁₄)₂, O(CH₂)mN(R₁₄)₂, C(═O)N(R₁₄)₂, and O(CH₂)mCOOR₁₄ where m is 1-5 and R₁₄ is H or lower alkyl;
or R₇ and R₈ together form a covalent bond;
or R₆ and R₇ together form ═Z, where Z is selected from the group consisting of O, S, and NH;
or X₁ and X₂ are each independently selected from the group consisting of —C(R₁₅)(R₁₆)—, O, S, NH, C═O, C═S, C═NH, SO, and SO₂, wherein R₁₅ and

R₁₆ are each independently selected from the group consisting of H, lower alkyl, hydroxy, lower alkoxy, halo, amino, aminoalkyl, alkylamino, nitro, heteroaryl, aryl, OC(═O)R₁₇, OC(═O)OR₁₇, OC(═O)N(R₁₇)₂, O(CH₂)mN(R₁₇)₂, C(═O)N(R₁₇)₂, and O(CH₂)mCOOR₁₇, where m is 1-5 and R₁₇ is H or lower alkyl;

or X₁ and X₂ together form —C═C—;
or X₃ is selected from the group consisting of O, S, NH to form a heterocycle, and (CH₂)ₚ where p is 1-2;
or a pharmaceutically acceptable salt or prodrug thereof.

A further aspect of the present invention is a pharmaceutical formulation comprising an active compound as described herein, in a pharmaceutically acceptable carrier (e.g., an aqueous carrier)

A still further aspect of the present invention is a method of treating a cancer, comprising administering to a human or animal subject in need thereof a treatment effective amount (e.g., an amount effective to treat, slow the progression of, etc.) of a compound as described above, and further described below. Examples of cancers that may be treated include, but are not limited to, skin cancer, lung cancer including small cell lung cancer and non-small cell lung cancer, testicular cancer, lymphoma, leukemia, Kaposi's sarcoma, esophageal cancer, stomach cancer, colon cancer, breast cancer, endometrial cancer, ovarian cancer, central nervous system cancer, liver cancer and prostate cancer.

A still further aspect of the invention is the use of an active compound or active agent as described herein for the preparation of a medicament for carrying out a method of treatment as described herein.

### DETAILED DESCRIPTION OF THE INVENTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like.

“Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like.

“Alkenyl,” as used herein, refers to a straight or branched chain hydrocarbon containing from 2 to 10 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 1 to 4 carbon atoms.

“Alkoxy,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like.

“Lower alkoxy” as used herein, is a subset of alkoxy and refers to a lower alkyl group, as defined herein, appended to the parent molecular moiety through an oxy group, as defined herein. Representative examples of lower alkoxy include, but are not limited to, methoxy, ethoxy, propoxy, 2-propoxy, butoxy, tert-butoxy, and the like.

“Alkylthio” as used herein refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Alkylogen” as used herein means alkyl or lower alkyl in which one, two, three or more (e.g., all) hydrogens thereon have been replaced with halo. Examples of alkylogen include but are not limited to trifluoromethyl, chloromethyl, 2-chloroethyl, 2-bromoethyl, and 2-iodoethyl. Alkylogens may also be referred to as haloalkyl or perhaloalkyl (e.g. fluoroalkyl; perfluoroalkyl).

“Cycloalkyl,” as used herein, refers to a saturated cyclic hydrocarbon group containing from 3 or 4 to 6 or 8 carbons. Representative examples of cycloalkyl include, cyclopropyl, cyclobutyl, cyclopentyl, cyclohexyl, cycloheptyl, and cyclooctyl.

“Heterocycle,” as used herein, refers to a monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizne, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like. Heterocycle groups of this invention can be substituted with 1, 2, or 3 substituents, such as substituents independently selected from alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, azido, arylalkoxy, arylalkoxycarbonyl, arylalkyl, aryloxy, carboxy, cyano, formyl, oxo, halo, haloalkyl, haloalkoxy, hydroxy, hydroxyalkyl, mercapto, nitro, sulfamyl, sulfo, sulfonate, —NR′R″ (wherein, R and R″ are independently selected from hydrogen, alkyl, alkylcarbonyl, aryl, arylalkyl and formyl), and —C(O)NRR′ (wherein, R and R′ are independently selected from hydrogen, alkyl, aryl, and arylalkyl).

“Aryl” as used herein refers to an aromatic species containing 1 to 5 aromatic rings, either fused or linked, and either unsubstituted or substituted with 1 or more typically selected from the group consisting of lower alkyl, modified lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxyl thio, mercapto, amino, imino, halo, cyano, nitro, nitroso, azido, carboxy, sulfide, sulfone, sulfoxy, phosphoryl, silyl, silyloxy, and boronyl. Typical aryl groups contain 1 to 3 fused aromatic rings, and more typical aryl groups contain 1 aromatic ring or 2 fused aromatic rings. Aromatic groups herein may or may not be heterocyclic.

“Heteroaryl” as used herein refers to an aryl, as defined herein, that is heterocyclic.

“Halo” as used herein refers to any halogen group, such as chloro, fluoro, bromo, or iodo.

“Oxo” as used herein, refers to a ═O moiety.

“Oxy,” as used herein, refers to a —O— moiety.

“Thio” as used herein refers to a —S— moiety.

“Amine” or “amino group” is intended to mean the radical —NH₂.

“Substituted amino” or “substituted amine” refers to an amino group, wherein one or two of the hydrogens is replaced by a suitable substituent. Disubstituted amines may have substituents that are bridging, i.e., form a heterocyclic ring structure that includes the amine nitrogen as the linking atom to the parent compound. Examples of substituted amino include but are not limited to alkylamino, dialkylamino, and heterocyclo (where the heterocyclo is linked to the parent compound by a nitrogen atom in the heterocyclic ring or heterocyclic ring system).

“Alkylamino” is intended to mean the radical —NHR′, where R′ is alkyl.

“Dialkylamino” is intended to mean the radical NR′R″, where R′ R″ are each independently an alkyl group.

“Aminoalkyl” refers to an alkyl substituent which is further substituted with one or more amino groups.

Dashed lines (e.g., ---) as used herein represent that the respective atoms are connected by either a single or double bond.

“Treat” or “treating” as used herein refers to any type of treatment that imparts a benefit to a patient afflicted with a disease, including improvement in the condition of the patient (e.g., in one or more symptoms), delay in the progression of the disease, prevention or delay of the onset of the disease, etc.

“Treatment effective amount” as used herein refers to an amount of the active compound effective to treat the disease, slow or delay the progression of the disease, prevent or delay of the onset of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

“Inhibit” as used herein means that a potential effect is partially or completely eliminated.

The present invention is concerned primarily with the treatment of human subjects, but may also be employed for the treatment of other animal subjects (i.e., mammals such as dogs, cats, horses, etc. or avians) for veterinary purposes. Mammals are preferred, with humans being particularly preferred.

### A. Active Compounds.

Active compounds of the present invention are described below, and may be formulated and used in the compositions and methods described below.

Active compounds of the invention include compounds of Formula I:

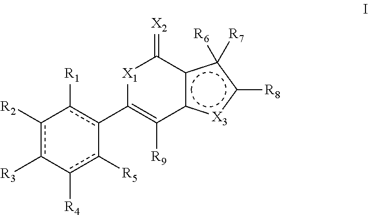

wherein:
R₁, R₂, R₃, R₄, R₅, R₆, R₇, R₈, and R₉ are each independently selected from the group consisting of H, lower alkyl, hydroxy, lower alkoxy, halo, amino, aminoalkyl, alkylamino, nitro, heteroaryl, aryl, OC(═O)R₁₄, OC(═O)OR₁₄, OC(═O)N(R₁₄)₂, O(CH₂)mN(R₁₄)₂, C(═O)N(R₁₄)₂, and O(CH₂)mCOOR₁₄ where m is 1-5 and R₁₄ is H or lower alkyl;
or R₇ and R₈ together form a covalent bond;
or R₆ and R₇ together form ═Z, where Z is selected from the group consisting of O, S, and NH;
or X₁ and X₂ are each independently selected from the group consisting of —C(R₁₅)(R₁₆)—, O, S, NH, C═O, C═S, C═NH, SO, and SO₂, wherein R₁₅ and R₁₆ are each independently selected from the group consisting of H, lower alkyl, hydroxy, lower alkoxy, halo, amino, aminoalkyl, alkylamino, nitro, heteroaryl, aryl, OC(═O)R₁₇, OC(═O)OR₁₇, OC(═O)N(R₁₇)₂, O(CH₂)mN(R₁₇)₂, C(═O)N(R₁₇)₂, and O(CH₂)mCOOR₁₇, where m is 1-5 and R₁₇ is H or lower alkyl;
or X₁ and X₂ together form —C═C—;
or X₃ is selected from the group consisting of O, S, NH to form a heterocycle, and (CH₂)ₚ where p is 1-2;
or a pharmaceutically acceptable salt or prodrug thereof.

In some embodiments of Formula I, X₁ is CH₂, S, NH, C═O, C═S, C═NH, SO, or SO₂.

In some embodiments of Formula I, X₂ is CH₂, O, S, NH, C═S, C═NH, SO, or SO₂. In other embodiments of Formula I, X₂ is C═O.

In some embodiments of Formula I, at least one of, or both of, X₁ and X₂ are CH₂. In some embodiments of Formula I, both X₁ and X₂ are CH₂. In some embodiments of Formula I, one of X₁ and X₂ is CH₂ and the other is not.

In some embodiments of Formula I, X₃ is CH₂, S, or NH.

In some embodiments of Formula I, X₁ is not O when X₂ is C═O.

In some embodiments of Formula I, R₇ and R₈ together form a covalent bond.

In some embodiments of Formula I, R₆ and R₇ together form ═Z, where Z is selected from the group consisting of O, S, and NH.

In some embodiments of Formula I, at least one of R₁, R₂, R₃, R₄, R₅, R₆, R₇, R₈, and R₉ is selected from the group consisting of lower alkoxy, halo, amino, aminoalkyl, alkylamino, nitro, heteroaryl, aryl, OC(═O)R₁₄, OC(═O)OR₁₄, OC(═O)N(R₁₄)₂, O(CH₂)mN(R₁₄)₂, C(═O)N(R₁₄)₂, and O(CH₂)mCOOR₁₄, where m is 1-5 and R₁₄ is H or lower alkyl.

### B. Formulations and Pharmaceutically Acceptable Salts.

The term “active agent” as used herein, includes the pharmaceutically acceptable salts of the compound of Formula I. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

Active agents used to prepare compositions for the present invention may alternatively be in the form of a pharmaceutically acceptable free base of active agent. Because the free base of the compound is less soluble than the salt, free base compositions are employed to provide more sustained release of active agent to the target area. Active agent present in the target area which has not gone into solution is not available to induce a physiological response, but serves as a depot of bioavailable drug which gradually goes into solution.

Active compounds of the invention include prodrugs thereof. A “prodrug” is a compound that, upon in vivo administration, is metabolized by one or more steps or processes or otherwise converted to the biologically, pharmaceutically or therapeutically active form of the compound of Formula I. To produce a prodrug, the pharmaceutically active compound is modified such that the active compound will be regenerated by metabolic processes (e.g., by conversion of a carboxylic acid to the corresponding C1-C4 ester or amide thereof). The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can produce prodrugs of the compound in accordance with known techniques (see, e.g., Nogrady (1985) Medicinal Chemistry A Biochemical Approach, Oxford University Press, New York, pages 388-392).

The compounds of the present invention are useful as pharmaceutically active agents and may be utilized in bulk form. More preferably, however, these compounds are formulated into pharmaceutical formulations for administration. Any of a number of suitable pharmaceutical formulations may be utilized as a vehicle for the administration of the compounds of the present invention.

The compounds of the present invention may be formulated for administration for the treatment of a variety of conditions hi the manufacture of a pharmaceutical formulation according to the invention, the compounds of the present invention and the physiologically acceptable salts thereof, or the acid derivatives of either (hereinafter referred to as the “active compound”) are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 95% by weight of the active compound. One or more of each of the active compounds may be incorporated in the formulations of the invention, which may be prepared by any of the well-known techniques of pharmacy consisting essentially of admixing the components, optionally including one or more accessory ingredients.

The formulations of the invention include those suitable for oral, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3:318 (1986)) and typically take the form of an optionally buffered aqueous solution of the active compound.

Suitable formulations comprise citrate or bis\tris buffer (pH 6) or ethanol/water and contain from 0.01 to 0.2M active ingredient.

### C. Methods of Use.

In addition to the compounds of the formulas described herein, the present invention also provides useful therapeutic methods. For example, the present invention provides a method of inducing cytotoxicity against tumor cells, or treating a cancer or tumor in a subject in need thereof.

Cancer cells which may be inhibited include cells from skin cancer, small cell lung cancer, non-small cell lung cancer, testicular cancer, lymphoma, leukemia, Kaposi's sarcoma, esophageal cancer, stomach cancer, colon cancer, breast cancer, endometrial cancer, ovarian cancer, central nervous system cancer, liver cancer and prostate cancer.

Subjects which may be treated using the methods of the present invention are typically human subjects although the methods of the present invention may be useful for veterinary purposes with other subjects, particularly mammalian subjects including, but not limited to, horses, cows, dogs, rabbits, fowl, sheep, and the like. As noted above, the present invention provides pharmaceutical formulations comprising the compounds of formulae described herein, or pharmaceutically acceptable salts thereof, in pharmaceutically acceptable carriers for any suitable route of administration, including but not limited to oral, rectal, topical, buccal, parenteral, intramuscular, intradermal, intravenous, and transdermal administration.

The therapeutically effective dosage of any specific compound will vary somewhat from compound to compound, patient to patient, and will depend upon the condition of the patient and the route of delivery. As a general proposition, a dosage from about 0.1 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight of the active base, including the cases where a salt is employed. Typically a dosage from about 0.5 mg/kg to about 5 mg/kg will be employed for intravenous or intramuscular administration. A dosage from about 10 mg/kg to about 50 mg/kg may be employed for oral administration.

The present invention is explained in greater detail in the following non-limiting examples.

### Example 1

### Substituted AFPO (6-aryl-4H-furo[3,2-c]pyran-4-one) derivatives

This example describes our initial design and synthesis of novel AFPO (6-aryl-4H-furo[3,2-c]pyran-4-one) derivatives, cytotoxicity evaluation against a panel of human cancer cell lines, and structure-activity relationships (SAR) of this new compound class. The goal is to generate and optimize AFPO (6-aryl-4H-furo[3,2-c]pyran-4-one) derivatives as promising clinical trial candidates for treating breast cancer.

Chemistry. All target compounds (8) were synthesized through a three to five-step sequence (Scheme 2). Various substituted esters (5) were reacted with a dianion intermediate generated from 2.5 eq of LDA, 1.0 eq ethyl acetoacetate, and 1.0 eq of TMEDA in THF to give triketoesters (6) as tautomeric mixtures. Pyrones (7) were prepared by heating 6 in a 170° C. oil bath at 5 mm Hg for 20 min to 1 h. A yellow solid formed immediately and was isolated by vacuum filtration and rinsed with 5% CH₂Cl₂ in diethyl ether. The compound obtained by this simple filtration could be used directly in the next step. (Douglas, C. J.; Sklenicka, H. M.; Shen, H. C.; Mathias, D. S.; Degen, S. J.; Golding, G. M.; Morgan, C. D.; Shih, R. A.; Mueller, K. L.; Seurer, L. M.; Johnson, E. W.; Hsung, R. P. Tetrahedron 1999, 55, 13683-13696). Target compounds 9-11, 14-17, 19, 21-22, 24-25, and 27-29 were obtained via a tandem alkylation/intramolecular Aldol reaction of 7. (Risitano, F., et al. Tetrahedron Lett. 2001, 42, 3503-3505). Removal of the methyl group in 11, 17, 19, 22, and 25 by BBr₃ gave 13, 18, 20, 23, and 26. Compound 12 was obtained by treatment of 13 with iodoethane under basic conditions (Scheme 2). Compound 25 was reacted with Lawesson's reagent to afford 30. (Boeckman, R. K., Jr., et al. Org. Lett. 2001, 3, 3647-3650).

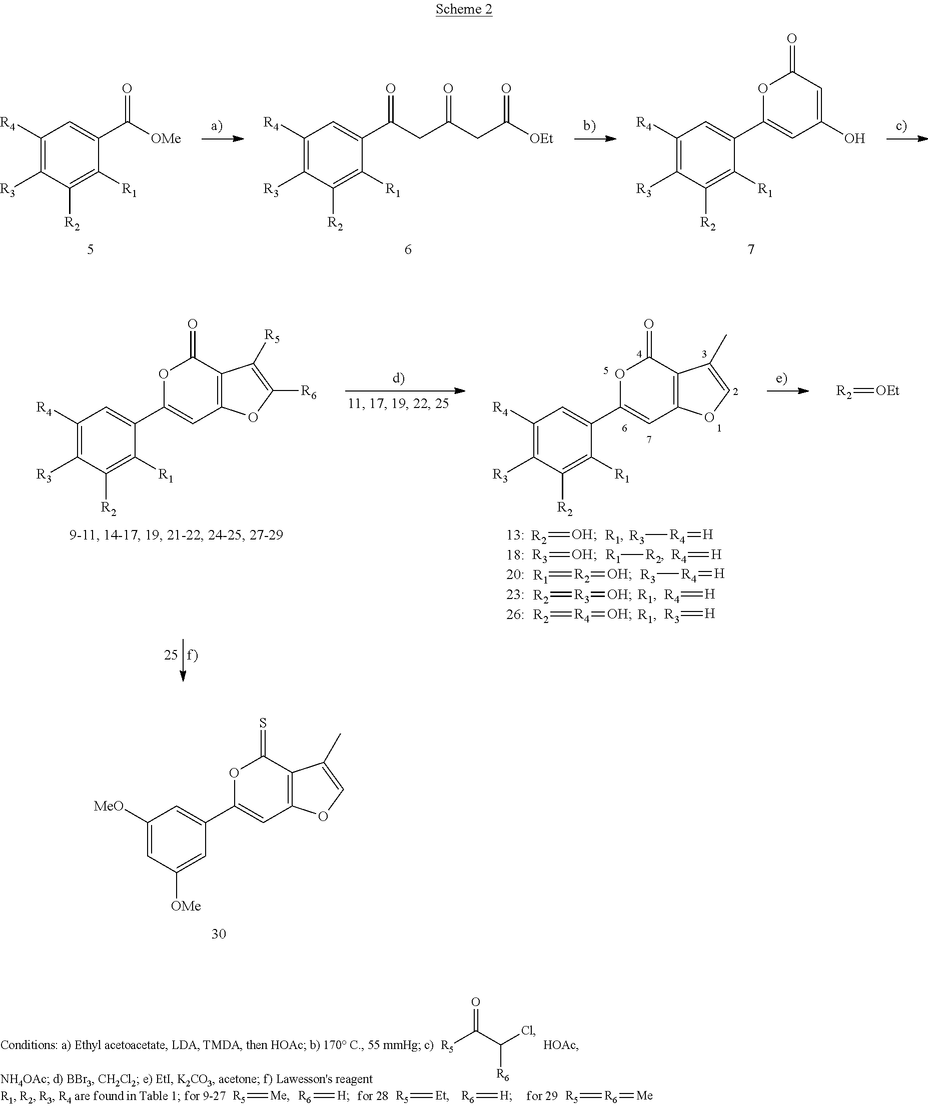

### Results and Discussion

Together with 1, the newly synthesized 6-phenyl-4H-furo[3,2-c]pyran-4-one analogs 9-30 were evaluated for in vitro anti-breast cancer activity against the SK-BR-3 (HER2+) human tumor cell line. Results from compounds 9-27 (Table 1) showed that different substituents around the phenyl ring were critical to the potency and selectivity. Modifications in the furopyranone ring system were also explored with 28-30 (Table 1). Selected active compounds with ED₅₀ values less than 4 μg/mL against SK-BR-3 were further examined against ZR-75-1 (ER+, HER2+), MDA-MB-231 (ER−), A549 (human lung cancer), DU145 (prostate cancer), KB (nasopharyngeal carcinoma), and KB-vin (vincristine resistant KB subline) cancer cell lines (Table 2).

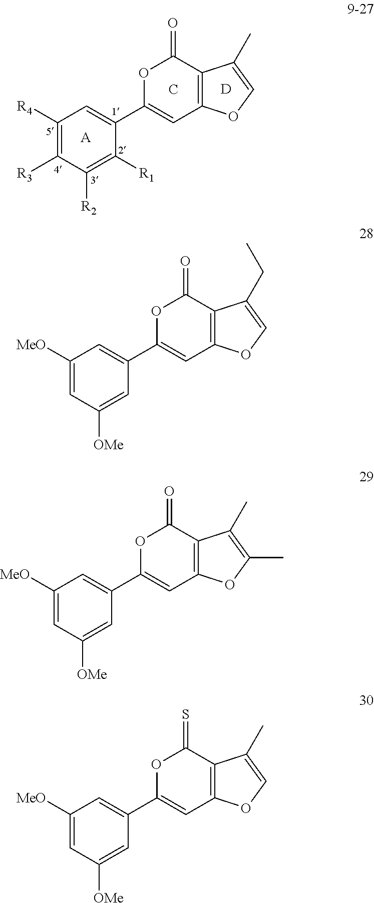

TABLE 1
Cytotoxicity of 9-27 Against SK-BR-3 Tumor Cell Line^{a}

| Compd | 2′ | 3′ | 4′ | 5′ | SK-BR-3 |
|---|---|---|---|---|---|
| 1 | — | — | — | — | 0.25 |
| 9 | H | H | H | H | 3.5 |
| 10 | H | Me | H | H | 0.36 |
| 11 | H | OMe | H | H | 0.66 |
| 12 | H | OEt | H | H | 0.18 |
| 13 | H | OH | H | H | 0.39 |
| 14 | H | F | H | H | 5.0 |
| 15 | H | H | F | H | 4.7 |
| 16 | H | H | Me | H | >20 |
| 17 | H | H | OMe | H | >20 |
| 18 | H | H | OH | H | 8.7 |
| 19 | OMe | OMe | H | H | 18.6 |
| 20 | OH | OH | H | H | 8.8 |
| 21 | H | Me | Me | H | 3.7 |
| 22 | H | OMe | OMe | H | 14.8 |
| 23 | H | OH | OH | H | 0.12 |
| 24 | H | Me | H | Me | 5.7 |
| 25 | H | OMe | H | OMe | 0.08 |
| 26 | H | OH | H | OH | 9.9 |
| 27 | H | OMe | OMe | OMe | 0.14 |
| 28 | — | — | — | — | 16.67 |
| 29 | — | — | — | — | >20 |
| 30 | — | — | — | — | >20 |

^{a}mean ED₅₀ (μg/mL), Standard error of independent determinations was less than 5%.

TABLE 2
Cytotoxicity of Selected Compounds against Tumor Cell Line Panel^{a}

| | | MDA-MB- | | | | |
|---|---|---|---|---|---|---|
| Compd | ZR-75-1 | 231 | A549 | DU145 | KB | KBvin |
| 1 | 0.25 | 10.0 | 14.3 | 15.4 | >10 | >20 |
| 10 | 1.5 | >20 | 12.9 | 5.9 | 10.5 | 9.6 |
| 11 | 1.4 | >10 | 14.9 | >20 | >20 | 18.2 |
| 12 | 0.60 | >10 | 15.6 | 20 | 17.4 | >20 |
| 13 | 0.21 | >10 | 16.6 | 20 | 15.8 | 14.6 |
| 23 | 0.31 | 5.9 | 5.0 | 6.6 | 5.2 | 6.0 |
| 25 | 8.8 | >20 | >20 | 18.7 | >20 | >20 |
| 27 | 9.2 | >10 | 20 | >20 | >20 | >20 |

^{a}See Table 1.

Structurally, both 1 and 10 have a methyl substituent at corresponding positions on their phenyl rings. Thus, the two compounds are identical, except that 10 has no ring-B. Interestingly, 10 showed potent activity with an ED₅₀ value of 0.36 μg/mL, which is slightly less active than 1. The unsubstituted analog 9 was less potent than either 1 or 10. Addition of methyl (10), methoxy (11), ethoxy (12), and hydroxyl (13) at the 3′-position of the phenyl ring increased activity against the SK-BR-3 cell line, compared with 9. The rank order of potency of the five compounds was 12>13>11>10>9. Compound 12, with a 3′-ethoxyphenyl ring, displayed slightly greater activity (ED₅₀ 0.18 μg/mL) than 1. In contrast, fluorine at the 3′-position (14), as well as 4′-position (15), led to somewhat decreased potency compared with the unsubstituted analog 9. Addition of methyl, methoxy, or hydroxyl at the phenyl 4′-position (16-18) reduced potency significantly. Compounds 19-26 and 27 are di- and tri-substituted derivatives, respectively, with one substituent always present at the phenyl 3′-position. Neither 2′,3′-disubstituted compound (19, 20) showed significant activity, leading us to speculate that a substituent in the 2′-position may have a steric effect on the orientation of the lactone ring and reduce the ligand-receptor interaction. Analogs with the same substituent at both the 3′- and 4′-positions showed increased potency relative to the corresponding 4′-monosubstituted analog (16 vs 21, 17 vs 22, 18 vs 23). Thus, alkyl, alkoxy, and hydroxy groups are favored at 3′-position, while they are not favored at the 4′-position. Comparison of 25 with 24 and 26 indicated that a methoxy group is favored at 5′-position, while methyl and hydroxy groups are not. Furthermore, the 3′,4′,5′-trimethoxy (27) and 3′,5′-dimethoxy (25) analogs showed dramatically enhanced potency compared with the 3′-methoxy compound (II), while the 3′,4′-dimethoxy (22) and 2′,3′-dimethoxy (19) analogs showed decreased potency. In fact, the 3′,5′-dimethoxy analog 25 (ED₅₀ 0.08 μg/mL) was the most active analog among the 19 substituted phenyl A-ring analogs (9-27). It was also approximately threefold more potent than 1.

We also investigated the cytotoxic activity of 28-30, which have a modified ring-C or -D, as shown in Table 1. Insertion of an ethyl (28) or two methyl (29) groups rather than a single methyl group on the furan, as well as bioisosteric replacement of sulfur (thiolactone 30) for oxygen in the lactone carbonyl led to greatly reduced or no anti-breast cancer activity (Table 1). More SAR studies of ring-C and -D are in progress and will be reported in a future publication.

To further examine the human tumor-tissue-type selectivity, active compounds 10-13, 23, 25, and 27 were tested against a limited but diverse panel of human cancer cell lines, using 1 as a positive control (Table 2). Compounds 10-13 and 23 displayed similar inhibition of the ZR-75-1 and SK− BR-3 cell lines. Interestingly, 25 and 27 showed very weak activity against ZR-75-1. Except for 23, all lead compounds had weak activity or no activity against MDA-MB-231 breast cancer or the remaining four cancer cell lines tested, which suggested high tumor-tissue-type selectivity. Furthermore, 23 showed only moderate inhibition. Importantly, 25 and 27 showed unique selectivity against the SK-BR-3 breast cancer cell line (HER2+), with approximately 100-250 fold differences compared with the other cancer cell lines tested. The unique selectivity of these novel lead compounds could be exploited to develop novel anti-breast cancer trials candidates and explore the mechanism(s) of action.

Compound 25 was tested independently against cell lines derived from normal breast tissue (MCF10A and 184A1) versus SK-BR-3 as a positive breast cancer cell line control, and results are shown in FIG. 2. The interpolated ED₅₀ values were 0.28, 4.8 and >10 μg/mL against SK-BR-3, 184A1, and MCF10A cells, respectively, showing that 25 is selective for a sub-set of breast cancer-derived cell lines and is significantly less active against normal breast-derived tissue.

### CONCLUSION

In conclusion, this study discovered a novel class of promising anti-breast cancer agents, substituted 6-phenyl-4H-furo[3,2-c]pyran-4-one derivatives. The ED₅₀ values of the two most potent analogs (25 and 27) against SK-BR-3 were 0.08 and 0.14 μg/mL, respectively. More importantly, 25 and 27 showed extremely high cancer cell line selectivity, being approximately 100- to 250-fold more potent against SK-BR-3 compared with six additional tested cancer cell lines. Furthermore, 25 displayed much greater potency against the SK-BR-3 breast cancer cell line compared with normal breast cell lines 184A1 and MCF10A. Preliminary SAR studies led to the following observations: (1) 3′-Methyl, methoxy, ethoxy, and hydroxy groups, but not a 3′-fluoro group, could increase potency; (2) Among disubstituted phenyl compounds, 2′-, 4′-, or 5′-methyl groups, 2′- or 4′-methoxy groups, and 5′-hydroxy groups decreased potency; while a 4′-hydroxy or 5′-methoxy group increased potency; (3) Current modifications in ring-C and -D were not preferred. The SAR profile established from the current study is different from that with the neo-tanshinlactone series, which is a four-ring system. Thus, skeletal planarity is not indispensable for the entire molecule, though it may be important to some extent. Focused studies will continue to develop promising novel analogs as clinical trials candidates for anti-breast cancer treatment.

### Experimental Section

Materials and Methods. Melting points were measured with a Fisher Johns melting apparatus without correction. ¹H NMR spectra were measured on a 300 MHz Varian Gemini 2000 spectrometer using TMS as internal standard. The solvent used was CDCl₃ unless indicated. Mass spectra were measured on a Shimadzu LC-MS2010 instrument. Thin-layer chromatography (TLC) and preparative TLC were performed on precoated silica gel GF plates purchased from Merck, Inc. Biotage Flash+ or Isco Companion systems were used for flash chromatography. Silica gel (200-400 mesh) from Aldrich, Inc. was used for column chromatography. All other chemicals were obtained from Aldrich, Inc. and Fisher, Inc.

Cell Growth Inhibition Assay. All stock cultures are grown in T-25 flasks. Freshly trypsinized cell suspensions were seeded in 96-well microliter plates with compounds added from DMSO-diluted stock. The plates were incubated for an additional 72 h after attachment and drug addition, and the assay was terminated by 10% TCA. Then, 0.4% SRB dye in 1% HOAc was added to stain the cells for 10 minutes. Unbound dye was removed by repeated washing with 1% HOAc and the plates were air dried. Bound stain was subsequently solved with 10 mM trizma base, and the absorbance read at 515 nm. Growth inhibition of 50% (GI₅₀) is calculated as the drug concentration, which caused a 50% reduction in the net protein increase in control cells during the drug incubation. The mean ED₅₀ is the concentration of agent that reduces cell growth by 50% under the experimental conditions and is the average from at least two independent determinations. Variation between replicates was no more than 5% of the mean. The following human tumor cell lines were used in the assay: A549 (non small cell lung cancer), ZR-75-1 (estrogen receptor positive breast cancer), MDA MB-231 (estrogen receptor negative breast cancer), SKBR-3 (estrogen receptor negative, HER-2 over-expressing breast cancer), KB (nasopharyngeal carcinoma), KB-VIN (vincristine-resistant KB subline). All cell lines were obtained from the Lineberger Cancer Center (UNC-CH) or from ATCC (Rockville, Md.). Cells propagated in RPMI-1640 supplemented with 10% FBS, penicillin (100 IU/mL), streptomycin (1 μg/mL), and amphotericin B (0.25 μg/mL), and were cultured at 37° C. in a humidified atmosphere of 95% air/5% CO₂.

### General Preparation of 9-11, 14-17, 19, 21-22, 24-25, 27-29

To a solution of 7 (1.04 mmol) in toluene (9 mL) was added a mixture of HOAc (0.30 ml, 5.20 mmol) and NH₄OAc (400 mg, 5.20 mmol) in EtOH (3 mL) and chloroacetone (0.42 mL, 5.20 mmol). The mixture was stirred for 30 min at rt, and then heated to 60° C. for 30 min. Subsequently, it was refluxed for 24 h. After cooling, the mixture was diluted with H₂O and extracted with EtOAc. The organic layer was dried over Na₂SO₄, filtered, and evaporated in vacuo. The residue was purified by column chromatography to give a white solid.

### Spectroscopic Data

### 3-Methyl-6-phenyl-4H-furo[3,2-c]pyran-4-one (9)

50% yield; mp 105-107° C.; ¹H NMR (300 MHz, CDCl₃, ppm): δ 2.33 (d, J=1.2 Hz, 3H, CH₃), 7.00 (s, 1H, OCH), 7.28-7.29 (m, 1H, C7-H), 7.43-7.46 (m, 3H, aromatic), 7.83-7.87 (m, 2H, aromatic); HRMS for (M⁺+H): calcd. 227.0708, found: 227.0696.

### 3-Methyl-6-m-tolyl-4H-furo[3,2-c]pyran-4-one (10)

52% yield; mp 135-137° C.; ¹H NMR (300 MHz, CDCl₃, ppm): δ 2.33 (d, J=1.2 Hz, 3H, CH₃), 2.42 (s, 3H, CH₃), 6.99 (s, 1H, C7-H), 7.23-7.36 (m, 3H, aromatic), 7.62 (d, J=7.2 Hz, 1H, aromatic), 7.69 (d, J=1.5 Hz, 1H, OCH); HRMS for (M⁺+H): calcd. 241.0865, found: 241.0851.

### 6-(3-Methoxyphenyl)-3-methyl-4H-furo[3,2-c]pyran-4-one (11)

44% yield; mp 119-121° C.; ¹H NMR (300 MHz, CDCl₃, ppm): δ 2.34 (d, J=1.5 Hz, 3H, CH₃), 3.87 (s, 3H, OCH₃), 6.96-7.00 (m, 2H), 7.29-7.44 (m, 4H); ¹³C NMR (300 MHz, CDCl₃, ppm): δ 8.52, 55.47, 93.64, 109.91, 110.68, 116.36, 117.82, 119.55, 129.91, 133.07, 140.76, 157.71, 159.39, 160.01, 161.93; HRMS for (M⁺+H): calcd. 257.0814, found: 257.0800.

### 6-(3-Fluorophenyl)-3-methyl-4H-furo[3,2-c]pyran-4-one (14)

34% yield; mp 158-160° C.; ¹H NMR (300 MHz, CDCl₃, ppm): δ 2.34 (d, J=1.5 Hz, 3H, CH₃), 7.01 (s, 1H, C7-H), 7.10-7.17 (m, 1H, aromatic), 7.31 (d, J=1.2 Hz, 1H, OCH), 7.39-7.46 (m, 1H, aromatic), 7.54-7.58 (m, 1H, aromatic), 7.61-7.65 (m, 1H, aromatic); HRMS for (M⁺+H): calcd. 245.0614, found: 245.0603.

### 6-(4-Fluorophenyl)-3-methyl-4H-furo[3,2-c]pyran-4-one (15)

52% yield; mp 175-177° C.; ¹H NMR (300 MHz, CDCl₃, ppm): δ 2.34 (d, J=1.5 Hz, 3H, CH₃), 6.94 (s, 1H, C7-H), 7.12-7.18 (m, 2H, aromatic), 7.29 (d, J=1.2 Hz, 1H, OCH), 7.82-7.87 (m, 2H, aromatic); HRMS for (M⁺+H): calcd. 245.0614, found: 245.0603.

### 3-Methyl-6-p-tolyl-4H-furo[3,2-c]pyran-4-one (16)

62% yield; mp 153-155° C.; ¹H NMR (300 MHz, CDCl₃, ppm): δ 2.33 (d, J=1.2 Hz, 3H, CH₃), 2.40 (s, 3H, CH₃), 6.95 (s, 1H, C7-H), 7.24-7.27 (m, 3H, aromatic & OCH), 7.74 (d, J=8.1 Hz, 2H, aromatic); HRMS for ([M+H]⁺): calcd. 241.0865, found: 241.0848.

### 6-(4-Methoxyphenyl)-3-methyl-4H-furo[3,2-c]pyran-4-one (17)

60% yield; mp 146-148° C.; ¹H NMR (300 MHz, CDCl₃, ppm): δ 2.33 (d, J=1.5 Hz, 3H, CH₃), 3.87 (s, 3H, OCH₃), 6.88 (s, 1H, 07-H), 6.97 (d, J=9.0 Hz, 2H, aromatic), 7.26 (d, J=1.5 Hz, 1H, OCH), 7.80 (d, J=9.0 Hz, 2H, aromatic); HRMS for ([M+H]⁺): calcd. 257.0808, found: 257.0816.

### 6-(2,3-Dimethoxyphenyl)-3-methyl-4H-furo[3,2-c]pyran-4-one (19)

40% yield; mp 111-113° C.; ¹H NMR (300 MHz, CDCl₃, ppm): δ 2.34 (d, J=1.2 Hz, 3H, CH₃), 3.87 (s, 3H, OCH₃), 3.92 (s, 3H, OCH₃), 7.00 (dd, J=1.2, 8.1 Hz, 1H, aromatic), 7.16 (t, J=8.1 Hz, 1H, aromatic), 7.34 (q, J=1.2 Hz, 1H, OCH), 7.48 (s, 1H, C7-H), 7.54 (dd, J=1.2, 8.1 Hz, 1H, aromatic); HRMS for (M⁺+H): calcd. 287.0919, found: 287.0906.

### 6-(3,4-Dimethylphenyl)-3-methyl-4H-furo[3,2-c]pyran-4-one (21)

67% yield; mp 182-184° C.; ¹H NMR (300 MHz, CDCl₃, ppm): δ 2.30 (s, 3H, CH₃) 2.32 (s, 3H, CH₃), 2.33 (d, J=1.5 Hz, 3H, CH₃), 6.95 (s, 1H, 07-H), 7.20 (d, J=8.1 Hz, 1H, aromatic), 7.26 (d, J=1.2 Hz, 1H, OCH), 7.57 (d, J=8.1 Hz, 1H, aromatic), 7.64 (s, 1H, aromatic); HRMS for ([M+H]⁺): calcd. 255.1016, found: 255.1010.

### 6-(3,4-Dimethoxyphenyl)-3-methyl-4H-furo[3,2-c]pyran-4-one (22)

83% yield; mp 154-156° C.; ¹H NMR (300 MHz, CDCl₃, ppm): δ 2.33 (d, J=1.5 Hz, 3H, CH₃), 3.94 (s, 3H, OCH₃), 3.98 (s, 3H, OCH₃), 6.90 (s, 1H, C7-H), 6.92 (d, J=8.7 Hz, 1H, aromatic), 7.27 (t, J=1.5 Hz, 1H, OCH), 7.34 (d, J=2.1 Hz, 1H, aromatic), 7.43 (dd, J=2.1, 8.4 Hz, 1H, aromatic); HRMS for (M⁺+H): calcd. 287.0919, found: 287.0900.

### 6-(3,5-Dimethylphenyl)-3-methyl-4H-furo[3,2-c]pyran-4-one (24)

30% yield; mp 171-173° C.; ¹H NMR (300 MHz, CDCl₃, ppm): δ 2.33 (d, J=0.9 Hz, 3H, CH₃), 2.37 (s, 6H, CH₃), 6.98 (s, 1H, 07-H), 7.07 (s, 1H, aromatic), 7.28 (d, J=0.9 Hz, 1H, OCH), 7.48 (s, 2H, aromatic); HRMS for (M⁺+H): calcd. 255.1021, found: 255.1010.

### 6-(3,5-Dimethoxyphenyl)-3-methyl-4H-furo[3,2-c]pyran-4-one (25)

38%, yield; mp 153-155° C.; ¹H NMR (300 MHz, CDCl₃, ppm): δ 2.34 (d, J=1.5 Hz, 3H, CH₃), 3.86 (s, 6H, OCH₃), 6.54 (t, J=2.1 Hz, 1H, C7-H), 6.98 (s, 1H, aromatic), 6.99 (d, J=2.7 Hz, 2H, aromatic), 7.30 (d, J=1.5 Hz, 1H, OCH); HRMS for (M⁺+H): calcd. 287.0919, found: 287.0898.

### 3-Methyl-6-(3,4,5-trimethoxyphenyl)-4H-furo[3,2-c]pyran-4-one (27)

40% yield; mp 201-203° C.; ¹H NMR (300 MHz, CDCl₃, ppm): δ 2.34 (d, J=1.5 Hz, 3H, CH₃), 3.91 (s, 3H, OCH₃), 3.95 (s, 6H, OCH₃), 6.94 (s, 1H, C7-H), 7.06 (s, 2H, aromatic), 7.29 (d, J=1.2 Hz, 1H, OCH); HRMS for (M⁺+H): calcd. 317.1025, found: 317.1037.

### 6-(3,5-Dimethoxyphenyl)-3-ethyl-4H-furo[3,2-c]pyran-4-one (28)

51% yield; mp 131-133° C.; ¹H NMR (300 MHz, CDCl₃, ppm): δ 1.31 (t, J=7.2 Hz, 3H, CH₂CH₃), 2.77 (q, J=7.5 Hz, 2H, CH₂CH₃), 3.86 (s, 6H, OCH₃), 6.54 (t, J=2.1 Hz, 1H, C7-H), 6.98 (d, J=2.1 Hz, 3H, aromatic), 7.30 (t, J=1.2 Hz, 1H, OCH); HRMS for (M⁺+H): calcd. 301.1076, found: 301.1057.

### 6-(3,5-Dimethoxyphenyl)-2,3-dimethyl-4H-furo[3,2-c]pyran-4-one (29)

12% yield; mp 163-165° C.; ¹H NMR (300 MHz, CDCl₃, ppm): δ 2.25 (d, J=0.6 Hz, 3H, CH₃), 2.33 (d, J=0.6 Hz, 3H, CH₃), 3.86 (s, 6H, OCH₃), 6.53 (t, J=2.1 Hz, 1H, aromatic), 6.93 (s, 1H, OCH), 6.97 (d, J=2.1 Hz, 2H, aromatic); HRMS for ([M+H]⁺): calcd. 301.1071, found: 301.1067.

### General Preparation of 13, 20, 23, and 26

To a solution of 13, 20, 23, or 26 (0.2 mmol) in DCM (3 ml) was added BBr₃ (0.6 ml, 0.6 mmol) dropwise at 0° C. The reaction mixture was stirred overnight. Water was added to quench the reaction. The solution was extracted with CHCl₃ and concentrated. The residue was purified by column chromatography to give a white solid.

### 6-(3-Hydroxyphenyl)-3-methyl-4H-furo[3,2-c]pyran-4-one (13)

78% yield; mp 225-227° C.; ¹H NMR (300 MHz, CD₃OD, ppm): δ 2.29 (d, J=1.5 Hz, 3H, CH₃), 6.86-6.90 (m, 1H, 07-H), 7.26-7.31 (m, 3H, aromatic & OCH), 7.35-7.39 (m, 1H, aromatic), 7.52 (dd, J=1.2, 2.7 Hz, 1H, aromatic); HRMS for (M⁺+H): calcd. 243.0657, found: 243.0659.

### 6-(4-Hydroxyphenyl)-3-methyl-4H-furo[3,2-c]pyran-4-one (18)

80% yield; mp 258-260° C.; ¹H NMR (300 MHz, CD₃OD, ppm): δ 2.27 (d, J=1.5 Hz, 3H, CH₃), 6.86 (d, J=9.0 Hz, 2H, aromatic) 7.18 (s, 1H, 07-H), 7.46 (d, J=1.2 Hz, 1H, OCH), 7.75 (d, J=9.3 Hz, 2H, aromatic); FIRMS for ([M+H]⁺): calcd. 243.0657, found: 243.0641.

### 6-(2,3-Dihydroxyphenyl)-3-methyl-4H-furo[3,2-c]pyran-4-one (20)

66% yield; mp 239-241° C.; ¹H NMR (300 MHz, CD₃OD, ppm): δ 2.28 (d, J=0.9 Hz, 3H, CH₃), 6.76 (t, J=1.5 Hz, 1H, aromatic), 6.86 (dd, J=1.5, 1.8 Hz, 1H, aromatic), 7.38 (dd, J=1.2, 1.5 Hz, 1H, aromatic), 7.48 (q, J=1.2 Hz, 1H, OCH), 7.75 (s, 1H, C7-H); HRMS for (M⁺+H): calcd. 259.0606, found: 259.0602.

### 6-(3,4-Dihydroxyphenyl)-3-methyl-4H-furo[3,2-c]pyran-4-one (23)

60% yield; mp 259-261° C.; ¹H NMR (300 MHz, CD₃OD, ppm): δ 2.69 (d, J=1.5 Hz, 3H, CH₃), 6.83 (d, J=7.8 Hz, 3H, 07-H), 7.12 (s, 1H, aromatic), 7.26-7.31 (m, 2H, aromatic), 7.46 (dd, J=1.2 Hz, 1H, OCH); HRMS for (M⁺−H): calcd. 257.0450, found: 257.0464.

### 6-(3,5-Dihydroxyphenyl)-3-methyl-4H-furo[3,2-c]pyran-4-one (26)

70% yield; mp>300° C.; ¹H NMR (300 MHz, CD₃OD, ppm): δ 2.86 (d, J=1.2 Hz, 3H, CH₃), 6.35 (t, J=2.1 Hz, 1H, aromatic), 6.80 (d, J=2.1 Hz, 2H, aromatic), 7.21 (s, 1H, 07-H), 7.51 (d, J=1.2 Hz, 1H, OCH); HRMS for ([M+H]⁺): calcd. 259.0601, found: 259.0594.

### 6-(3-Ethoxyphenyl)-3-methyl-4H-furo[3,2-c]pyran-4-one (12)

To a mixture of 13 (212 mg, 1.00 mmol), K₂CO₃ (300 mg, 2.17 mmol) in actone (8 mL) was added iodoethane (0.4 mL, 5.00 mmol). The mixture was stirred for 12 h, The mixture was concentrated and diluted with H₂O and extracted with EtOAc. The organic layer was dried over Na₂SO₄, filtered, and evaporated. The residue was purified by column chromatography to give a white solid.

35% yield; mp 128-130° C.; ¹H NMR (300 MHz, CDCl₃, ppm): δ 1.45 (t, J=6.9 Hz, 3H, CH₂CH₃), 2.33 (d, J=1.5 Hz, 3H, CH₃), 4.10 (d, J=7.2 Hz, 2H, CH₂CH₃), 6.95-6.96 (m, 1H, aromatic), 6.98 (s, 1H, C7-H), 7.29 (d, J=1.5 Hz, 1H, OCH), 7.32-7.43 (m, 3H, aromatic); HRMS for ([M+H]⁺); calcd. 271.0965, found: 271.0962.

### 6-(3,5-Dimethoxyphenyl)-3-methyl-4H-furo[3,2-c]pyran-4-thione (30)

A mixture of 25 (0.1 mmol) and Lawesson's reagent (81 mg, 0.2 mmol) in dry toluene (5 mL) was heated to reflux for 12 h. Toluene was removed and the red residue was dissolved in EtOAc and partitioned with H₂O. The organic phase was separated and dried over Na₂SO₄. Removal of solvent in vacuo afforded an oily, residue which was purified by column chromatography resulting in a yellow solid.

60% yield; mp 147-149° C.; ¹H NMR (300 MHz, CDCl₃, ppm): δ 2.46 (d, J=1.5 Hz, 3H, CH₃), 3.87 (s, 6H, OCH₃), 6.57 (t, J=2.1 Hz, 1H, C7-H), 7.02 (s, 1H, aromatic), 7.03 (s, 1H, aromatic), 7.17 (s, 1H, aromatic), 7.32 (d, J=1.5 Hz, 1H, OCH); HRMS for (M⁺+H): calcd. 303.0691, found: 303.

### Methodology of MTT Assay

The MTT assay was used to access the in vitro anticancer activity of 25 against two normal breast cancer cell lines 184A1 and MCF10A (CRL-10317) purchased from ATCC (Rockville, Md.) and using SK-BR-3 as a positive control. Cells were seeded into 96 well plates at a density of 5000 cells per well in the recommended growth medium. The drug was dissolved in DMSO. The drug was added into wells after overnight incubation. After 72 h of incubation at 37° C. in 5% CO₂, 20 μL of MTT [3-(4,5-dimethylthiazol-2-yl)-2,5-diphenyl tetrazolium bromide] reagent was added to each well and incubation continued for two h. The amount of formazan product was measured at an OD of 570 nM using a plate-reader.

1. A compound of Formula I: 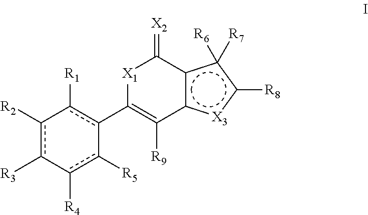
wherein:
R1, R2, R3, R4, R5, R6, R7, R8, and R9 are each independently selected from the group consisting of H, lower alkyl, hydroxy, lower alkoxy, halo, amino, aminoalkyl, alkylamino, nitro, heteroaryl, aryl, OC(═O)R14, OC(═O)OR14, OC(═O)N(R14)₂, O(CH₂)mN(R14)2, C(═O)N(R14)2, and O(CH₂)mCOOR14 where m is 1-5 and R14 is H or lower alkyl;
or R7 and R8 together form a covalent bond;
or R6 and R7 together form ═Z, where Z is selected from the group consisting of O, S, and NH;
or X1 and X2 are each independently selected from the group consisting of —C(R15)(R16)-, O, S, NH, C═O, C═S, C═NH, SO, and SO2, wherein R15 and R16 are each independently selected from the group consisting of H, lower alkyl, hydroxy, lower alkoxy, halo, amino, aminoalkyl, alkylamino, nitro, heteroaryl, aryl, OC(═O)R₁₇, OC(═O)OR₁₇, OC(═O)N(R17)2, O(CH₂)mN(R17)2, C(═O)N(R17)2, and O(CH2)mCOOR17, where m is 1-5 and R₁₇ is H or lower alkyl;
or X1 and X2 together form —C═C—;
or X₃ is selected from the group consisting of O, S, NH to form a heterocycle, and (CH₂)ₚ where p is 1-2;
or a pharmaceutically acceptable salt or prodrug thereof.

2. The compound of claim 1, wherein X₁ is CH₂, S, NH, C═O, C═S, C═NH, SO, or SO₂.

3. The compound of claim 1, wherein X₁ is O.

4. The compound of claim 1, wherein X₂ is CH₂, O, S, NH, C═S, C═NH, SO, or SO₂.

5. The compound of claim 1, wherein X₂ is C═O.

6. The compound of claim 1, wherein X₁ and X₂ are CH₂.

7. The compound of claim 1, wherein X₁ and X₂ together form —C═C—.

8. The compound of claim 1, wherein both X₁ and X₂ are CH₂.

9. The compound of claim 1, wherein one of X₁ and X₂ is CH₂ and the other is not.

10. The compound of claim 1, wherein X₃ is CH₂, S, or NH.

11. The compound of claim 1, wherein X₁ is not O when X₂ is C═O.

12. The compound of claim 1, wherein R₇ and R₈ together form a covalent bond.

13. The compound of claim 1, wherein R₆ and R₇ together form ═Z, where Z is selected from the group consisting of O, S, and NH.

14. The compound of claim 1, wherein at least one of R₁, R₂, R₃, R₄, R₅, R₆, R₇, R₈, and R₉ is selected from the group consisting of lower alkoxy, halo, amino, aminoalkyl, alkylamino, nitro, heteroaryl, aryl, OC(═O)R₁₄, OC(═O)OR₁₄, OC(═O)N(R₁₄)₂, O(CH₂)mN(R₁₄)₂, C(═O)N(R₁₄)₂, and O(CH₂)mCOOR₁₄, where m is 1-5 and R₁₄ is H or lower alkyl.

15. A pharmaceutical formulation comprising a compound of claim 1 in a pharmaceutically acceptable carrier.

16. The pharmaceutical formulation of claim 15, wherein said carrier is an aqueous carrier.

17. A method of treating a cancer, comprising administering to a human or animal subject in need thereof a treatment effective amount of a compound of claim 1.

18. The method of claim 17, wherein said cancer is selected from the group consisting of skin cancer, lung cancer, testicular cancer, lymphoma, leukemia, Kaposi's sarcoma, esophageal cancer, stomach cancer, colon cancer, breast cancer, endometrial cancer, ovarian cancer, central nervous system cancer, liver cancer and prostate cancer.

19. The method of claim 17, wherein said cancer is breast cancer.

20. The use of a compound of claim 1 for treating cancer, or for the preparation of a medicament for treating cancer.

21-22. (canceled)
